(19) United States
(12) Patent Application Publication
(10) Pub. No.: US 2010/0160790 A1
(43) Pub. Date: Jun. 24, 2010

(54) WEARABLE PHOTOACOUSTIC VASCULAR IMAGING SYSTEM

(75) Inventor: EFFIONG ETUKUDO IBOK, SUNNYVALE, CA (US)

Correspondence Address: THE TRAVIS BUSINESS GROUP, INC., 1005 E. HOMESTEAD RD., SUNNYVALE, CA, 94087, US

(73) Assignees: EFFIONG ETUKUDO IBOKSUNNYVALE, SUNNYVALE, CA (US); THE TRAVIS BUSINESS GROUP, INC, SUNNYVALE, CA (US)

(21) Appl. No.: 12/558,114

(22) Filed: Sep. 11, 2009

Related U.S. Application Data
(60) Provisional application No. 61/203,367, filed on Dec. 23, 2008.

(51) Int. Cl. A61B 6/00

(52) U.S. Cl. 600/476

(57) ABSTRACT

A non-invasive wearable imaging apparatus for vascular detection employing photo-acoustic principle is disclosed. Additionally, a wearable micro-display component is also described. The method includes pulsed laser as an energy source at a wavelength range at which both the blood and the adjoining tissue absorb the light and convert the energy into kinetic energy which heats the tissue. The temperature of the tissue rises and falls in sympathy with the frequency of the impinging light. The alternative heating and cooling of the illuminated region causes alternating sympathetic expansion and contraction of the region with corresponding rising and falling of the tissue surface and of the air in contact with the surface, thereby generating sound waves. A sensor enables the differentiation between the venous and surrounding tissues from which imaging of the venous tissue is extracted. In addition to the acoustic sensors, the sympathetic rising and falling of the tissue surface are converted into color characteristic signals representative of the relative displacement of the tissue surface. Additionally, the differential heating of the tissues enables the visual detection of the venous tissues by appropriate filter lenses. These embodiments also present miniaturization of the generation, imaging and display components, enabling the detection of the blood vessels in a wearable form factor.

### FIELD OF INVENTION

The present invention relates to the area of vascular imaging and detection for all forms of medical treatment requiring access of patients' veins either for delivery of medicine or the extraction of blood.

### BACKGROUND OF INVENTION

Vascular detection is a critical component of any medical delivery regimen. Whether in the extraction of blood for analysis, the intravenous delivery of medication or the injection of contrast dyes for imaging, vein detection is critical to that component of medical care. The lack of clarity in vein location and identification quite often leads to guess-work in needle insertion by the medical practitioner. An exercise which for the patient is quite often a matter of painful experimentation, and for the practitioner an embarrassing practice of trial and error. Proficiency is quite often a skill acquired from years of practice tinged with luck. Additionally, some fast action medications require intra-venous (I-V) delivery. A fast unequivocal identification of a vein for the I-V insertion could mean the difference between life and death for the patient. This invention removes the guess work from this critical medical regimen.

Vascular imaging has been an ever-present and needed capability of medical delivery since the 1920's. Generally, the various adaptations of Computerized Tomography (CT), x-rays, ultrasound and magnetic resonance (MR) have been used in venous and arterial imaging to various degrees of success and clarity. Recent advances in CT scanning for example Spiral CT Angiography is non-invasive and has allowed doctors to see blood vessels without the need for invasive procedure.

In U.S. Pat. No. 6,592,522 Bjaerum et al disclose an ultrasound machine that generates a color representation of internal moving structures and displays them on the screen. Adler discloses in U.S. Pat. No. 6,692,430 an invasive catheter based imaging apparatus designed for insertion into a blood vessel via a guide wire. In U.S. Pat. No. 6,662,040 Henrichs et al disclose a photoacoustic imaging device comprising the injection of a contrast agent and the exposing of said body to irradiation of from RF to X-rays. For the purpose of this application this methodology would be inappropriate in many of its scopes from size to contamination of the blood. Lewis et al in U.S. Pat. No. 5,314,679 disclose a magnetic resonance imaging system employing biodegradable contrast agent of super-paramagnetic metal oxide crystals. In U.S. Pat. No. 5,337,749 Shimizu et al disclose a magnetic resonance vascular imaging methodology.

However, all these methods of vascular imaging involve very expensive and large radiology equipment which while widely in use is neither portable nor practical in the level of venous or arterial imaging and detection for the purpose described herein.

### SCOPE OF INVENTION

This invention addresses the limitations of detecting vasculature in general and sub-dermal vasculature in particular for analytical evaluations, blood extractions and other intravenous procedures.

### BRIEF SUMMARY OF INVENTION

A method of vascular imaging using photo-acoustic principle is described. Additionally, a wearable micro-display component is also described. The method includes pulsed laser as an energy source at a wavelength range at which both the blood and the adjoining tissue absorb the light and convert the energy into kinetic energy which heats the tissue. The temperature of the tissue rises and falls in sympathy with the frequency of the impinging light. The alternative heating and cooling of the illuminated region causes alternating sympathetic expansion and contraction of the region with corresponding rising and falling of the tissue surface and of the air in contact with the surface, thereby generating sound waves. A sensor enables the differentiation between the venous and surrounding tissues from which imaging of the venous tissue is extracted. In addition to the acoustic sensors, the sympathetic rising and falling of the tissue surface are converted into color characteristic signals representative of the relative displacement of the tissue surface. Additionally, the differential heating of the tissues enables the visual detection of the venous tissues by appropriate filter lenses. These embodiments also present miniaturization of the generation, imaging and display components, enabling the detection of the blood vessels in a wearable form factor.

### BRIEF DESCRIPTION OF DRAWINGS

For understanding the present invention, reference is made to the following drawings in the “DETAILED DESCRIPTION OF THE INVENTION”. In the drawings:

FIG. 1 is the flow chart schematics of the principle of the invention.

FIG. 2 shows the schematics of the laser generation and modulation unit.

FIG. 3. Shows the schematics of the optical fiber connection unit between the generation and the sensing units.

FIG. 4a shows a schematic of the pressure sensor array strap unit.

FIG. 4b shows a plan view image of the Ultrasound Pressure Detection Matrix.

FIG. 5 shows a schematic of signal processor and switching units.

FIG. 6 is a schematic of the imaging unit showing display processors and image display unit.

FIG. 7 shows schematics of the transmission of the generated image signals wirelessly from the display processor to the portable image display unit.

FIG. 8 is a schematic of image micro-display unit with embedded wireless receiver.

FIG. 9 Narrow band filter lens for unassisted photo-acoustic irradiated vasculature detection.

### DETAILED DESCRIPTION OF INVENTION

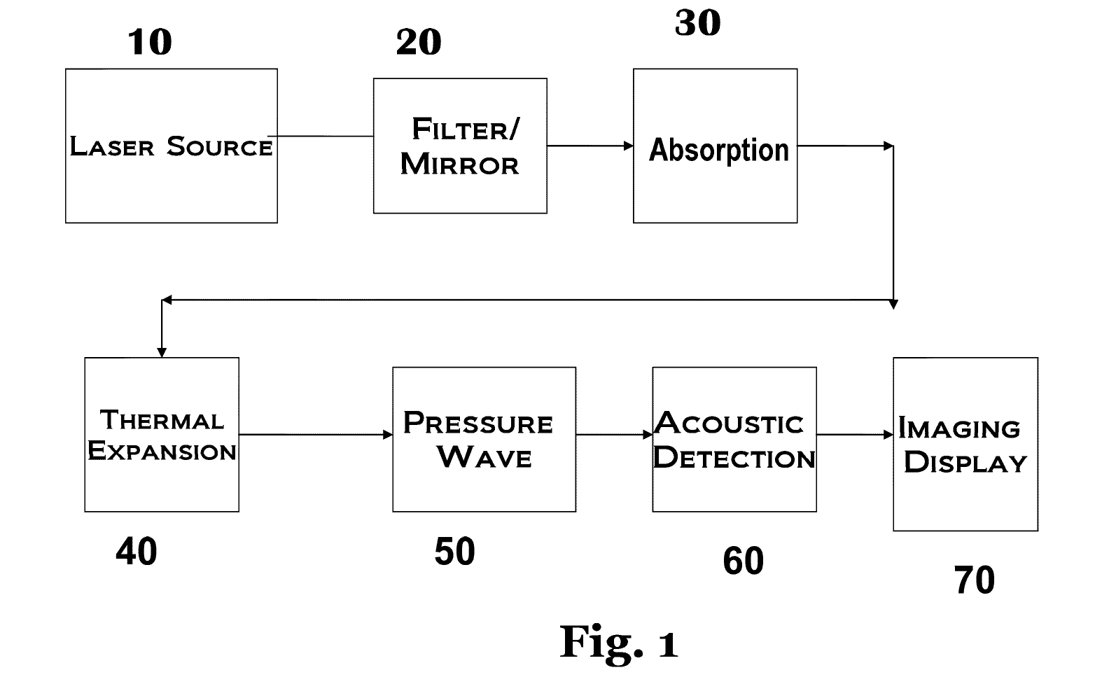

FIG. 1 shows the schematics of the general principle of the invention. Excitation light or laser pulses in the visible or NIR optical wavelengths are generated from any semiconductor laser source 10. The wavelengths are such as are transparent to body tissue, typically 600 to 1300 nm. The signals are transmitted through the optical fiber line 15 to the tissue where absorption takes place, 20. The large variation in the optical absorption and scattering of the various tissues, particularly hemoglobin is exploited to provide a broad optical contrast and heating differential 30. The differential in heating coupled with a differential in coefficient of expansion of the adjoining tissues result in a variation in the thermal expansion of the tissues, 40. The pulsed irradiation generates periodic expansion and contraction of the irradiated tissue in sympathy with the frequency of the irradiation and contrasting components of the irradiated tissue. If the temperature rises in any portion of the irradiated volume than that volume component can expand, a local pressure increase (a wave) is generated, 50. The pressure wave can be considered a sound wave and be detected by any acoustic detection method, 60. This generation of sound waves by optical irradiation is generally referred to as a photo-acoustic technique. The signal is sent out through an image processor and observed through any appropriate display system, 70.

We now explore the individual components of the imaging system hereby disclosed. While there are several photoacoustic systems in the literature, including some disclosed for venous and arterial imaging, the equipment are generally, large, cumbersome, or table top, PC-based systems.

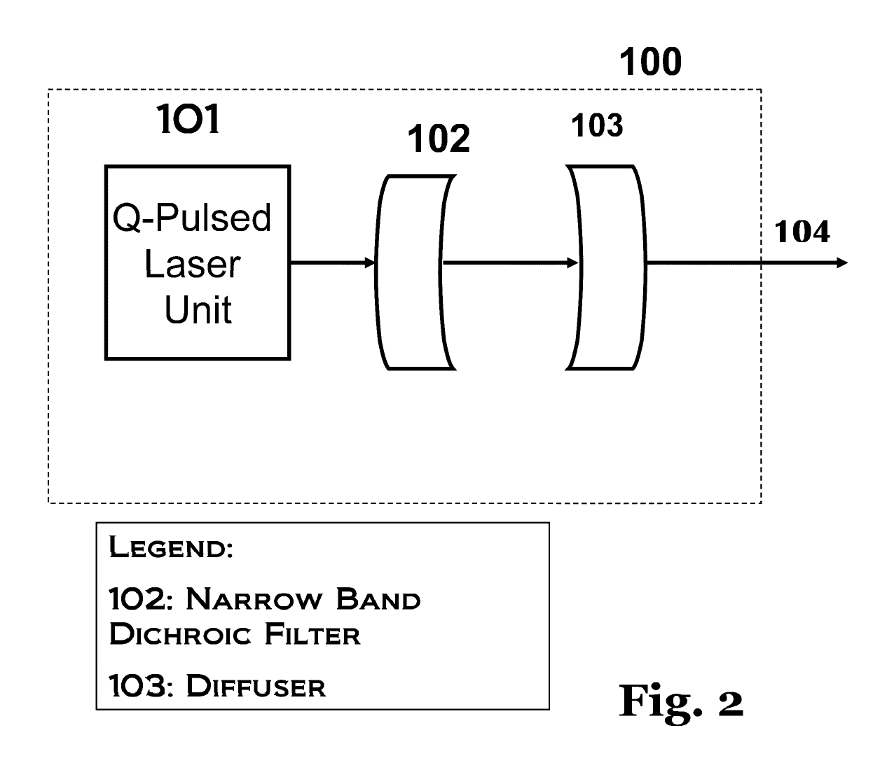

FIG. 2 shows the laser and mirror unit, 101 as described in one of the embodiments of the invention. In this embodiment, Nb-YAG laser source with continuous frequency in the range 400-1500 nm is emitted per known art. The radiation unit may be a semiconductor source. The emitting radiation may be controlled so that the emission pulse may be in the range of 10 to 200 MHz. In yet another embodiment of this invention, the radiation pulsing may be affected by a Q-pulse controlling device.

In as yet another embodiment of this invention, the emitted radiation is passed through a narrow beam dichroic filter 102 that transmits only the desired radiation wavelength that would maximize absorption in the blood vessel relative to the adjoining tissue. The transmitted radiation is further passed through a diffuser (103) to enlarge the footprint of the laser output beam to between 1 and 10 mm² to ensure an incident surface fluence of less than 0.1 J/cm² —the maximum permissible exposure for nanosecond NIR irradiation of the skin.

In as yet another embodiment, to enhance the miniaturization of the system, are all done in-situ and on wafer at the integration of the laser unit. Advantage is taken of the modulation of the effective refractive index of multilayer silicon dielectric films such as silicon nitride, oxynitride and oxide. The nitride with an RI range of 1.7 to 2.2 and the silicon oxynitride with an RI range of 1.6 to 2.2. The films are deposited by plasma enhanced chemical deposition (PECVD) during the laser source integration to thicknesses of between 200 Å to 1 μm. The refractive index modulation provides convex or concave lenses as the case may be depending on the location and properties of the different layers. The layer distribution can also be chosen to effect absorption or reflectivity of particular wavelengths of incident light.

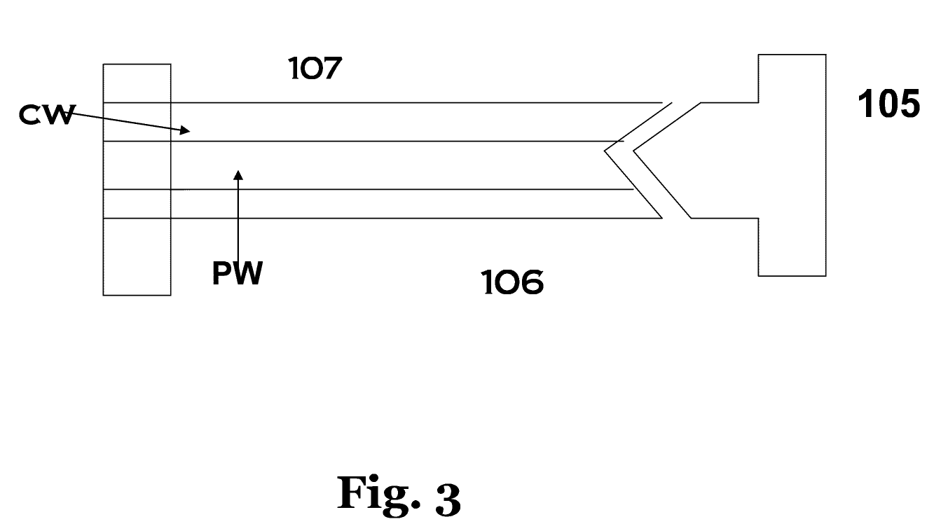

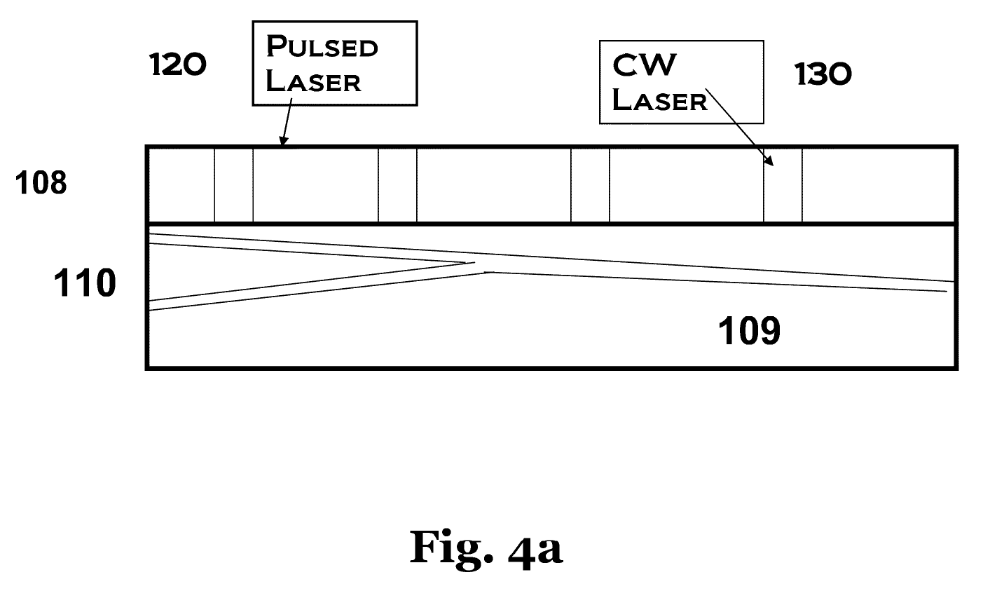

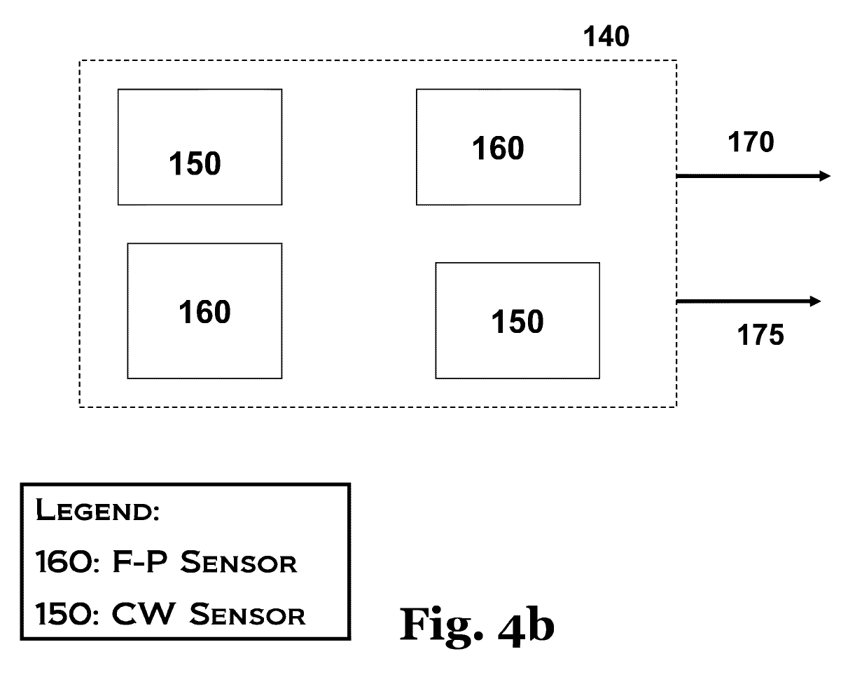

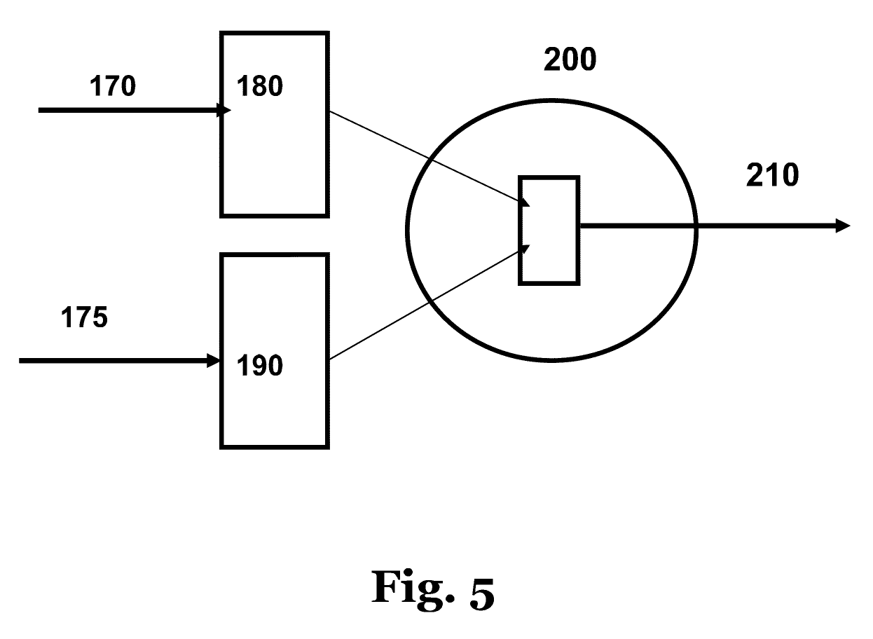

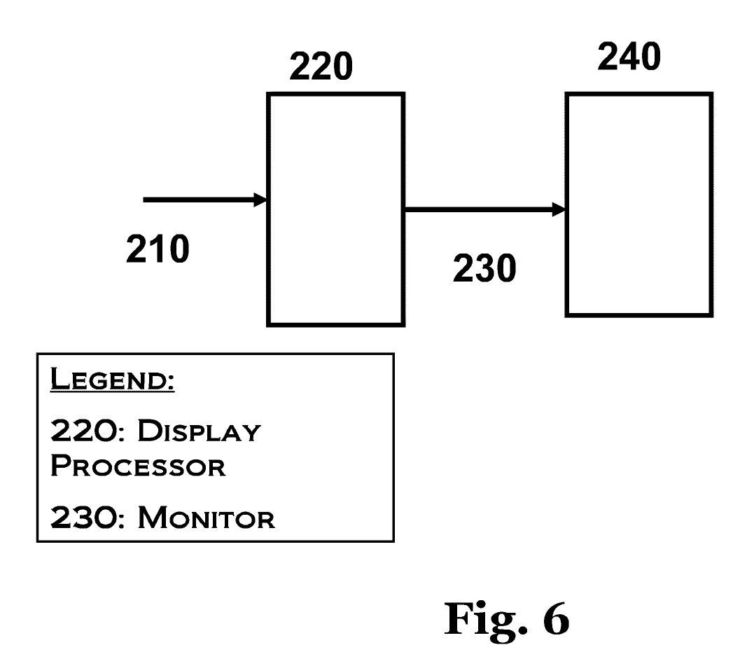

The pulsed laser is transmitted through optical fiber 105 (FIG. 3) from the laser unit to the sensor array 140 (FIG. 4). Concurrent with the pulsed laser beam 106 is a stream of continuous wave (CW) laser 107 for signal interrogation. The sensor array 140 is integrated into a cuff band 108 overlaying the tissue 109 and the veins 110 in question. The impact of the pulsed radiation 120 causes differential heating of the arterial and venous blood vessels relative to the adjoining tissue. This causes differential rate of expansion of the vessels relative to the adjoining tissue. The expansion and contraction in sympathy with the pulsed irradiation creates a pressure wave at the tissue surface adjacent to the sensor array that is picked up by the interrogating CW laser beam sensor 130 and the ultrasound F-P sensor matrix 140. The signals as received from the CW sensor 170 and the Fabry-Perot (F-P) sensor 175 are passed into signal processors 180 and 190 respectively (FIG. 5) that converts the signals into relative displacements parameters. The displacement parametric signal outputs are fed into an operator selectable switch 200. The parametric signal output 210 is passed into a display processor 220 (FIG. 6) which converts the received signal into a color characteristic signal representative of the displacement of the moving structure, in this case the surface of the tissue moving in sympathy with the blood vessel. The color characteristic signals 230 are sent on to a display monitor 240 on which the blood vessels are monitored and accessed.

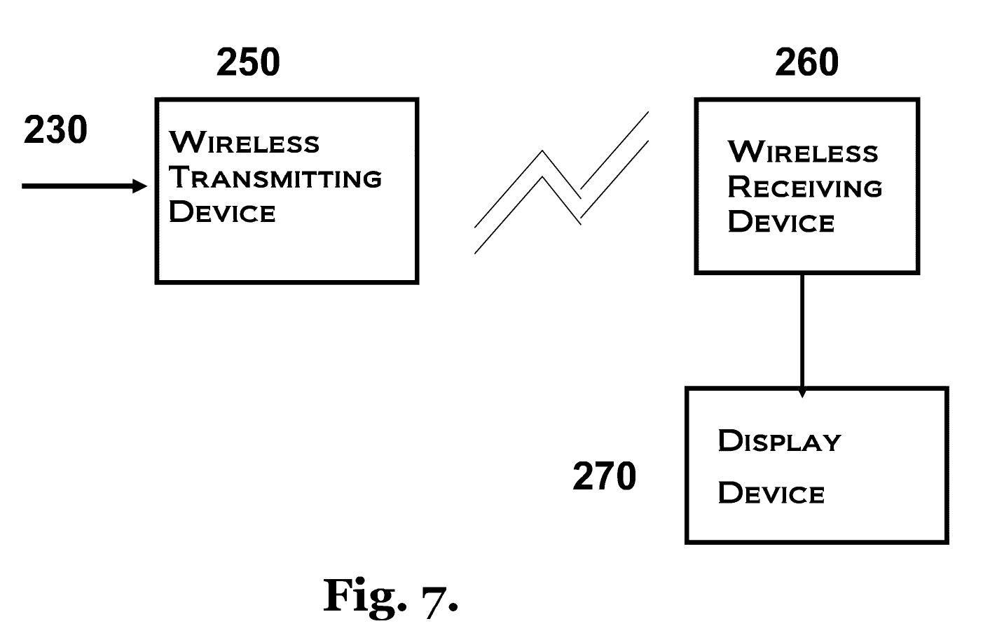

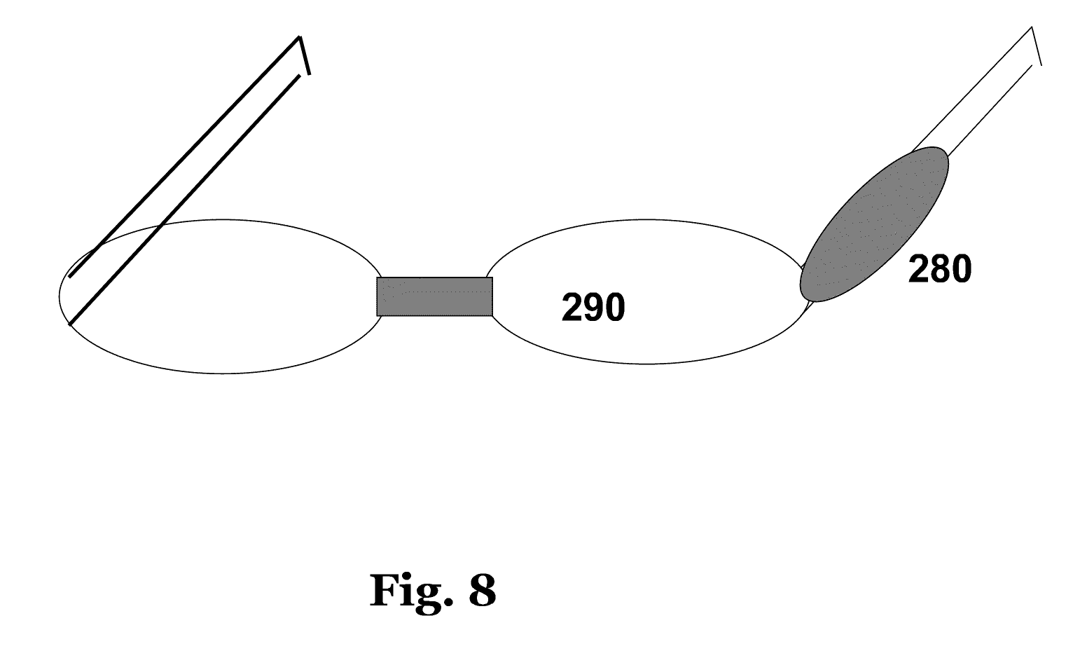

In another embodiment of this invention, the signals from the display processor 230 are sent to an RF transmitting device 250 (FIG. 7) which transmits the signal wirelessly to a portable display device 270 via an RF receiving device 260. The RF transmitting and receiving devices may be embedded Bluetooth components 280 (FIG. 8) and the portable display device may be an on-iris or other wearable micro-display system 290.

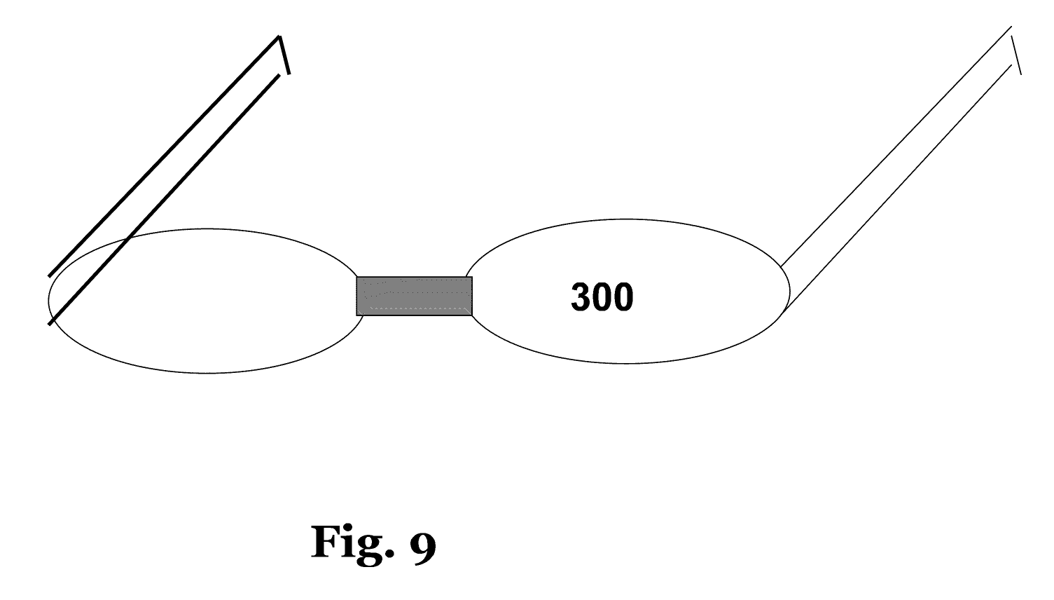

In yet another embodiment of the invention, advantage is taken of the difference in both the coefficient of expansion and the heat capacity of the hemoglobin in the blood vessels relative to the adjacent tissue. The differential increase in temperature and displacement in the sub-dermal blood vessels enables the detection of the blood vessels with an appropriate lens filter 300 (FIG. 9).

It is understood that the presentation of these steps in this disclosure is not exhaustive. Only the preferred embodiments of the invention and but a few of the examples of its versatility are shown and described in the present disclosure. It should be readily apparent to those of ordinary skill in the art that the invention is capable of use in various other combinations, environments and applications and is capable of changes or modifications within the scope of the inventive concept as expressed herein. These changes and modifications may be made without departing from the spirit and scope of the invention as set forth in the appended claims.

1. A method of vascular detection by wearable photoacoustic device wherein:
A light or laser source emits pulsed radiation in wavelength range of 600-1300 nm and said radiation passed through a narrow beam dichroic filter which transmits only the desired wavelength for optimum absorption in the blood vessels relative to adjoining tissue, and
Said radiation being passed through an optical fiber unit and said transmitted radiation being further passed a diffuser to enlarge the footprint of the radiation output.
Said output being transmitted to a sensor array.
Said sensor-array being integrated into a cuff-band overlaying the tissue and veins.
Said impacting pulsed radiation creating a pressure wave detected by an FP sensor
Detected signal being passed into a signal processor and
Said signal processor converting said signals into relative displacement parameters and
Said displacement parameters fed into a display processor for conversion into a color characteristic representative of displacement and
Said color characteristic signals sent to display monitor.

2. A method of claim 1 wherein a continuous wave (CW) laser is transmitted concurrently with the pulsed laser through the optical fiber for the purpose signal interrogation.

3. A method of claim 1 wherein the displacement parametric signals are processed through an operator selectable switch.

4. A method of claim 1 wherein the signals from the display processors are sent to an RF transmitting device

5. A method of claim 1 wherein the said RF transmitting device transmits said signals to a portable (wearable) display device via an RF receiving device.

6. A method of claim 5 wherein said RF devices may be embedded Bluetooth components.

7. A method of claim 5 wherein the portable display device may be an on-iris micro-display system.

8. A method of claim 1 wherein the laser source may be any semiconductor laser.

9. A method of claim 1 wherein the laser source is a Nb-YAg laser.

10. A method of claim 1 wherein the CW laser is of frequency in the range 300-1600 nm.

11. A method of claim 1 wherein the impacting radiation footprint is between 1-10 mm.

12. A method of claim 1 wherein the surface fluence of the said impact radiation is less than 0.5 J/m²

13. A method of claim 1 wherein the emission pulse is in the range of 10-200 MHz.

14. A method of claim 1 wherein the irradiation pulsing is by any Q-pulse controller.

15. A method of detecting vasculature optical differentiation wherein the thermal differentiation is by frequency filter lens capable of detecting the minor thermal variation between the veins and the adjoining tissue.

16. A method of claim 1 wherein the unit miniaturization is by on-wafer integration of the laser unit.

17. A method of claim 1 wherein the frequency filter is by on-wafer integration by the deposition of PECVD Silicon Dioxide filling defined by lithography and etch of said lens.

18. A method of claim 1 wherein the diffuser unit is by on-wafer integration by multilayer deposition and definition of Silicon Oxynitride layers of varying refractive indices between 1.6 and 2.2.

19. A method of claim 1 wherein the detector sensor is F-P.

20. A method of claim 1 wherein the detection sensor is CW lasers.
